(19) United States
(12) Patent Application Publication
(10) Pub. No.: US 2004/0180898 A1
(43) Pub. Date: Sep. 16, 2004

(54) Processes for preparing imidazoquinoxalinones, heterocyclic-substituted imidazopyrazinones, imidazoquinoxalines and heterocyclic-substituted imidazopyrazines

(76) Inventors: Bang-Chi Chen, Plainsboro, NJ (US); Joseph E. Sundeen, Yardley, PA (US); Mark S. Bednarz, Yardley, PA (US); Ping Chen, Belle Mead, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/378,536

(22) Filed: Mar. 3, 2003

(51) Int. Cl. A61K 31/498; C07D 487/14

(52) U.S. Cl. 514/250; 544/346

(57) ABSTRACT

Novel processes for the preparation of imidazoquinoxalinones, heterocyclic-substituted imidazopyrazinones, imidazoquinoxalines and heterocyclic-substituted imidazopyrazines are described.

### FIELD OF THE INVENTION

The present invention relates to processes for the preparation of imidazoquinoxalinones, heterocyclic-substituted imidazoquinoxalinones, imidazoquinoxalines and heterocyclic-substituted imidazopyrazines, which are useful for the treatment of cardiovascular disease, central nervous system disease and immunologic disorders.

### BACKGROUND OF THE INVENTION

Imidazoquinoxalinones are a class of hetereocyclic compounds useful as cardiovascular agents (Davey, D. D.; Erhardt, P. W.; Cantor, E. H.; Greenberg, S. S.; Ingebretsen, W. R.; Wiggins, J. J. Med. Chem. 1991, 34, 2671; Davey, D. D. EP 400583; Lee, T. D.; Brown, R. E. U.S. Pat. No. 4,440,929) and central nervous system agents (Jacobsen, E. J.; Stelzer, L. S.; Belonga, K. L.; Carter, D. B.; Im, W. B.; Sethy, V. H.; Tang, A. H.; VonVoigtlander, P. F.; Petke, J. D. J. Med. Chem. 1996, 39, 3820; TenBrink, R. E.; Jacobsen, E. J.; Hester, J. B., Jr.; Skaletzky, L. L. WO 9317025; Jacobsen, E. J. WO 9204350; Holger, C. H.; Watjen, F. U.S. Pat. No. 5,075,304; Watjen, F.; Hansen, H. C. U.S. Pat. No. 4,999,353; Hansen, H. C.; Watjen, F. U.S. Pat. No. 4,999,354). Imidazoquinoxalinones and heterocyclic-substituted imidazopyrazinones are also key intermediates in the synthesis of, respectively, imidazoquinoxaline protein tyrosine kinase inhibitors and heterocyclic-substituted imidazopyrazine protein tyrosine kinase inhibitors useful in the treatment, including prevention and therapy, of protein kinase-associated disorders such as immunologic disorders. Imidazoquinoxaline protein tyrosine kinase inhibitors are disclosed in U.S. Pat. Nos. 6,235,740 and 6,239,133. Heterocyclic-substituted imidazopyrazine protein tyrosine kinase inhibitors are disclosed in U.S. Pat. No. 5,990,109. The entire disclosure of each of these patents is herein incorporated by reference.

Previously, imidazoquinoxalinones were prepared via four different methods. In the first method, 2-halonitrobenzene is reacted with imidazole to give 2-(imidazolyl)nitrobenzene. Reduction of 2-(imidazolyl)nitrobenzene, followed by treating the resulting 2-(imidazolyl)aniline with 1,1′-carbonyldiimidazole, affords imidazoquinoxalinone product (Davey, D. D.; Erhardt, P. W.; Cantor, E. H.; Greenberg, S. S.; Ingebretsen, W. R.; Wiggins, J. J. Med. Chem. 1991, 34, 2671; Ohmori, J.; Shimizu-Sasamata, M.; Okada, M.; Sakamoto, S. J. Med. Chem. 1997, 40, 2053; Davey, D. D. EP 400583). When imidazole-4,5-dicarboxylate was used as the starting material, reduction of the 2-nitro group resulted in direct formation of imidazoquinoxalinone product (Lee, T. D.; Brown, R. E. U.S. Pat. No. 4,440,929). This method, however, is limited by regioselectivity when an unsymmetrically substituted imidazole is used. In addition, 2-fluoronitrobenzenes are usually required for a satisfactory aromatic nucleophilic substitution, and the fluoride by-product generated in this reaction is an environmental hazard in large scale production.

In the second method, 2-haloaniline was condensed with imidazole-4-carboxylic acid dimer to give an amide intermediate which upon heating afforded imidazoquinoxalinone product (U.S. Pat. No. 6,235,740). Although this method avoids the regioselectivity encountered in the first method, high temperature is required for the cyclization reaction due to the absence of the nitro activating group, and relatively electron rich 2-haloanilines do not work. Furthermore 2-fluoroanilines are required for a satisfactory aromatic nucleophilic substitution, and again the fluoride by-product generated in this reaction is an environmental hazard in large scale production.

In the third method, a 2-nitroaniline was reduced to 1,2-phenylenediamine, which was condensed with oxalyl chloride to quinoxalin-2,3-dione. Conversion of quinoxalin-2,3-dione to 3-phosphoryloxy-quinoxalin-2-one and subsequent reaction of arylmethyl isocyanide gave imidazoquinoxalinone product (Jacobsen, E. J.; Stelzer, L. S.; Belonga, K. L.; Carter, D. B.; Im, W. B.; Sethy, V. H.; Tang, A. H.; VonVoigtlander, P. F.; Petke, J. D. J. Med. Chem. 1996, 39, 3820; Jacobsen, E. J. WO 9204350; TenBrink, R. E.; Jacobsen, E. J.; Hester, J. B., Jr.; Skaletzky, L. L. WO 9317025). Like the first method, a regioselectivity problem exists in the conversion of quinoxalin-2,3-dione to 3-phosphoryloxy-quinoxalin-2-one when an unsymmetrically substituted quinoxalin-2,3-dione is used as the starting material.

In the fourth method, 1,2-phenylenediamine was condensed with glyoxylate to give quinoxalin-2-one. After protection of the 1-nitrogen, the resulting intermediate was reacted with tosylmethylisocyanide to give imidazoquinoxalinone product (U.S. Pat. No. 6,235,740). Again, a regioselectivity problem arises when an unsymmetrically substituted 1,2-phenylenediamine is used as the starting material. In addition to two extra steps (protection and deprotection) involved in this method, problems exist in the protection of the 1-nitrogen due to the competing side reaction, O-protection, making this method not amenable to large scale preparation due to the difficulty of separating the O-protected by-product from the desired N-protected product.

Preparation of 1,5-diarylsubstitued imidazoles using tosylmethylisocyanide was reported (Massa, C.; DiSanto, R.; Costi, R.; Artico, M. J. Heterocycl. Chem. 1993, 30, 749).

Preparation of N-aryl glyoxylate imines was also reported (Borrione, E.; Prato, M.; Scorrano, G.; Stivanello, M.; Lucchini, V. J. Heterocycl. Chem. 1988, 25, 1831).

Preparation of 1-arylimidazole-5-carboxylates has recently been reported (Chen, B.-C.; Bednarz, M. S.; Zhao, R.; Sundeen, J. E.; Chen, P.; Shen, Z.; Skoumbourdis, A. P.; Barrish, J. C.), and subsequent conversion to imidazoquinoxalines has also been described (Chen, P.; Norris, D.; Iwanowicz, E. J.; Spergel, S. H.; Lin, J.; Gu, H. H.; Shen, Z.; Wityak, J.; Lin, T.-A.; Pang, S.; de Fex, H.; Pitt, S.; Shen, D. R.; Doweyko, A. M.; Bassolino, D. A.; Roberge, J. Y.; Poss, M. A.; Chen, B.-C.; Schieven, G. L.; Barrish, J. C. Bioorg. Med. Chem. Lett. 2002, 12, 1361.)

### SUMMARY OF THE INVENTION

The present invention is directed to a process for preparing a compound of formula I,

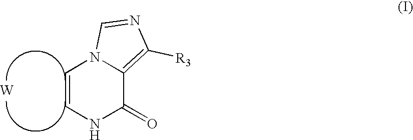

wherein

W, together with the atoms to which it is bonded, is a 4-15 membered monocyclic or bicyclic ring system optionally including up to 4 heteroatoms selected from N, O or S, and wherein a carbon atom in the said ring system is optionally substituted with oxo, and wherein W is optionally substituted with 1-3 substituents selected from the group consisting of halogen, trifluoromethyl, trifluoromethoxy, alkyl, alkenyl, alkynyl, cyano, ORₐ, SRₐ, NR_{b}R_{c}, NR_{b}SO₂Rₐ, SO₂Rₐ, SO₂NR_{b}R_{c}, CO₂Rₐ, C(═O)Rₐ, C(═O)NR_{b}R_{c}, OC(═O)Rₐ, OC(═O)NR_{b}R_{c}, NR_{b}C(═O)ORₐ, NR_{d}C(═O)NR_{b}R_{c}, NR_{b}C(═O)Rₐ, cycloalkyl, heterocyclo, aryl, and heteroaryl, wherein Rₐ is alkyl, alkenyl, alkynyl, cycloalkyl, heterocyclo, aryl or heteroaryl, R_{b}, R_{c}, and R_{d} are independently H, alkyl, cycloalkyl, heterocyclo, aryl, or heteroaryl, said R_{b} and R_{c} together with the N to which they are bonded optionally form a heterocyclic ring;

R₃ is selected from the group consisting of H, C₁-C₄ alkyl, cycloalkyl, heterocyclo, aryl and heteroaryl;

comprising reacting a compound of Formula V,

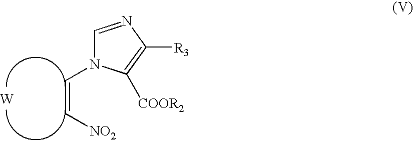

wherein W and R₃ are as defined hereinabove, and R₂ is C₁-C₄ alkyl,

with a reducing reagent selected from the group consisting of iron, zinc, sodium hydrosulfite and sodium hydrosulfite hydrate, in the presence of an acid, with heating.

A preferred process for making the compound of formula I, comprises the process wherein W is aryl or heteroaryl.

A more preferred process for making the compound of formula I, comprises the process wherein W is phenyl or pyridyl.

This invention is also directed to a process for making the compound of formula I,

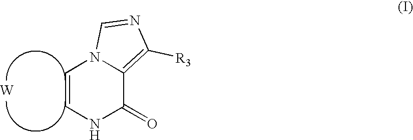

wherein W and R₃ are as defined above,

comprising:

(a) reacting a compound of formula III,

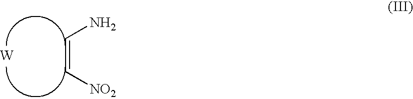

wherein W is as defined above,

with a compound of formula X,

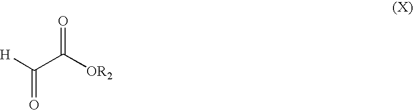

wherein R₂ is C₁-C₄ alkyl,

and a compound of formula XI,

R₁OH (IX)

wherein R₁ is C₁-C₄ alkyl.

to produce a compound of formula IV;

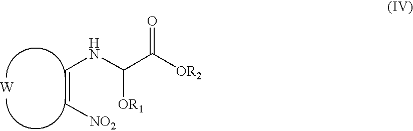

(b) reacting the compound of formula IV with a compound of formula XII,

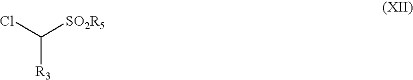

wherein R₃ is as defined above, and R₅ is aryl, in the presence of a base to produce a compound of formula V; and

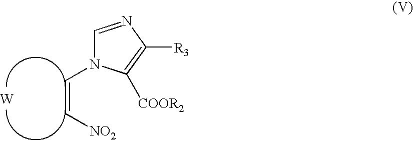

(c) reacting the compound of formula V with a reducing reagent selected from the group consisting of iron, zinc, sodium hydrosulfite, and sodium hydrosulfite hydrate, in the presence of an acid, with heating.

A preferred process for making the compound of formula I, comprises the process wherein W is aryl or heteroaryl.

A more preferred process for making the compound of formula I, comprises the process wherein W is phenyl or pyridyl.

This invention is also directed to a process for making the compound of formula I, further comprising converting the compound of formula I to a compound of formula XIV,

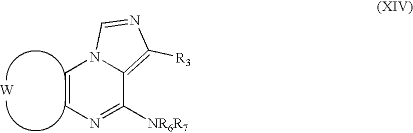

wherein W and R₃ are defined as above; R₆ and R₇ are independently H, alkyl, cycloalkyl, heterocyclo, aryl, heteroaryl, or R₆ and R₇ together with the N to which they are bonded optionally form a heterocyclic ring.

This invention is also directed to a process for making the compound of formula I, further comprising converting the compound of formula I to a compound of formula XV,

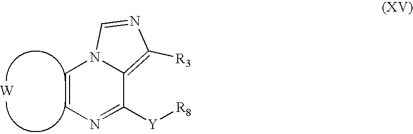

wherein W and R₃ are defined as above; Y is a bond, O, or S; R₈ is alkyl, alkenyl, alkynyl, cycloalkyl, heterocyclo, aryl, or heteroaryl.

This invention is also directed to a process for preparing a compound of formula II,

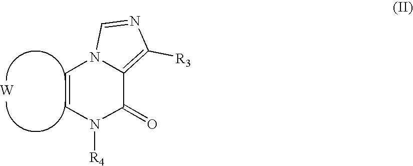

wherein

W, together with the atoms to which it is bonded, is a 4-15 membered monocyclic or bicyclic ring system optionally including up to 4 heteroatoms selected from N, O or S, and wherein a carbon atom in the said ring system is optionally substituted with oxo, and wherein W is optionally subsutituted with 1-3 substituents selected from the group consisting of halogen, trifluoromethyl, trifluoromethoxy, alkyl, alkenyl, alkynyl, cyano, ORₐ, SRₐ, NR_{b}R_{c}, NR_{b}SO₂Rₐ, SO₂Rₐ, SO₂NR_{b}R_{c}, CO₂Rₐ, C(═O)Rₐ, C(═O)NR_{b}R_{c}, OC(═O)Rₐ, OC(═O)NR_{b}R_{c}, NR_{b}C(═O)ORₐ, NR_{d}C(═O)NR_{b}R_{c}, NR_{b}C(═O)Rₐ, cycloalkyl, heterocyclo, aryl, and heteroaryl, wherein Rₐ is alkyl, alkenyl, alkynyl, cycloalkyl, heterocyclo, aryl or heteroaryl, R_{b}, R_{c} and R_{d} are independently H, alkyl, cycloalkyl, heterocyclo, aryl, or heteroaryl, said R_{b} and R_{c} together with the N to which they are bonded optionally form a heterocyclic ring;

R₃ and R₄ are, independently, H, alkyl, cycloalkyl, heterocyclo, aryl, or heteroaryl;

comprising:

(a) reacting a compound of formula VI,

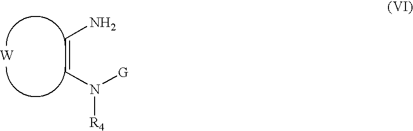

wherein W and R₄ are as defined hereinabove, G is an amine protecting group,

with a compound of formula X,

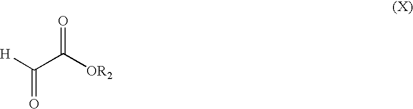

wherein R₂ is C₁-C₄ alkyl,

and a compound of formula XI,

R₁OH (XI)

wherein R₁ is C₁-C₄ alkyl,

to produce a compound of formula VII;

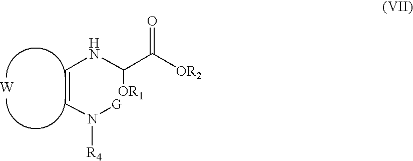

(b) reacting the compound of formula VII with a compound of formula XII,

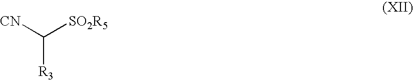

wherein R₃ is as defined hereinabove, and R₅ is aryl, in the presence of a base to produce a compound of formula VIII; and

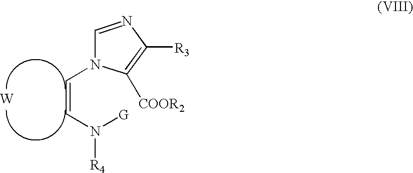

(c) deprotecting the compound of formula VIII to give a compound of formula IX; and

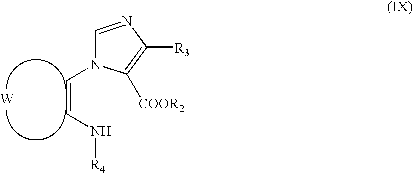

(d) reacting the compound of formula IX with a base while heating.

A preferred process for making the compound of formula II, comprises the process wherein W is aryl or heteroaryl.

A more preferred process for making the compound of formula II, comprises the process wherein W is phenyl or pyridyl.

This invention is also directed to a process for making the compound of formula II, further comprising converting the compound of formula II wherein R₄ is H, to a compound of formula XIV,

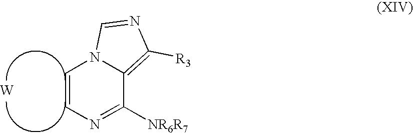

wherein W, R₃, R₆ and R₇ are defined as above.

This invention is also directed to a process for making the compound of formula II, further comprising converting the compound of formula II wherein R₄ is H, to a compound of formula XV,

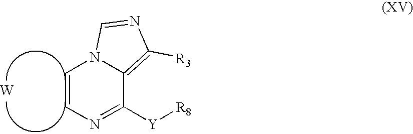

wherein W, R₃, Y and R₈ are defined as above.

### DETAILED DESCRIPTION OF THE INVENTION

This invention provides novel, more efficient and cost effective processes for the preparation imidazoquinoxalinones, heterocyclic-substituted imidazopyrazinones, or salts thereof. These processes use more readily available starting materials, avoid the generation of environmentally hazardous fluoride by-product, and are more convenient and amenable for large scale preparation. The processes of the present invention are shown in Schemes 1 and 2. A compound depicted in Schemes 1 and 2 is herein referred to by the Roman numeral under the compound in the Schemes, eg. “compound I” or “compound of the formula I.” In Schemes 1 and 2, imidazoquinoxalinones and heterocyclic-substituted imidazopyrazinones are compounds of the formulas I and II.

Solvents, temperatures, pressures, and other reaction conditions not specified may readily be selected by one of ordinary skill in the art. All documents cited are incorporated herein by reference in their entirety. Starting materials for the processes of the present invention are prepared by methods described herein, commercially available, or readily prepared by one of ordinary skill in the art.

Listed below are definitions of various terms used in the specifications and claims to describe the present invention.

The term “alkyl” refers to an optionally substituted straight or branched chain hydrocarbon groups having 1 to 12 carbon atoms, preferably 1 to 8 carbon atoms. Lower alkyl groups, that is, alkyl groups of 1 to 4 carbon atoms, are most preferred.

An “optionally substituted” organic group is one that is unsubstituted or substituted with one, two, or three substituents. The optional substituent may be selected from the group consisting of halogen, trifluoromethyl, trifluoromethoxy, alkenyl, alkynyl, nitro, cyano, oxo (═O), ORₐ, SRₐ, NR_{b}R_{c}, NR_{b}SO₂Rₐ, SO₂Rₐ, SO₂NR_{b}R_{c}, CO₂Rₐ, C(═O)Rₐ, C(═O)NR_{b}R_{c}, OC(═O)Rₐ, OC(═O)NR_{b}R_{c}, NR_{b}C(═O)ORₐ, NR_{d}C(═O)NR_{b}R_{c}, NR_{b}C(═O)Rₐ, cycloalkyl, heterocyclo, aryl and heteroaryl, wherein Rₐ is hydrogen, alkyl, alkenyl, alkynyl, cycloalkyl, heterocyclo, aryl or heteroaryl, R_{b}, R_{c} and R_{d} are independently hydrogen, alkyl, cycloalkyl, heterocyclo, aryl, or heteroaryl, or said R_{b} and R_{c} together with the N to which they are bonded optionally form a heterocyclic ring.

The term “alkenyl” refers to straight or branched chain hydrocarbon groups having 2 to 12 carbon atoms and at least one double bond. Alkenyl groups of 2 to 6 carbon atoms and having one double bond are most preferred.

The term “alkynyl” refers to straight or branched chain hydrocarbon groups having 2 to 12 carbon atoms and at least one triple bond. Alkynyl groups of 2 to 6 carbon atoms and having one triple bond are most preferred.

When a subscript is used as in C₁₋₄alkyl, the subscript refers to the number of carbon atoms the group may contain. Thus, for example, “C₁₋₄alkyl” refers to straight and branched chain alkyl groups with one to four carbon atoms, such as methyl, ethyl, n-propyl, isopropyl, n-butyl, t-butyl, and so forth.

The term “halo” or “halogen” refers to chloro, bromo, fluoro and iodo.

The term “cycloalkyl” refers to an optionally substituted, saturated cyclic hydrocarbon ring system.

The term “aryl” refers to monocyclic or bicyclic aromatic hydrocarbons having 6 to 12 carbon atoms in the ring portion, such as phenyl, biphenyl, 1-naphthyl, and 2-naphthyl, which may be optionally substituted. The term “phenyl” also includes optionally substituted phenyl.

The term “heterocyclo” refers to optionally substituted non-aromatic 3 to 7 membered monocyclic groups, 7 to 11 membered bicyclic groups, and 10 to 15 membered tricyclic groups, in which at least one of the rings has at least one heteroatom (O, S or N). Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing bicyclic and tricyclic groups may contain only carbon atoms and may be saturated, partially saturated, or unsaturated. The nitrogen and sulfur atoms may optionally be oxidized and the nitrogen atoms may optionally be quaternized. The heterocyclo group may be attached at any available nitrogen or carbon atom.

Thus, exemplary heterocyclo groups include, without limitation:

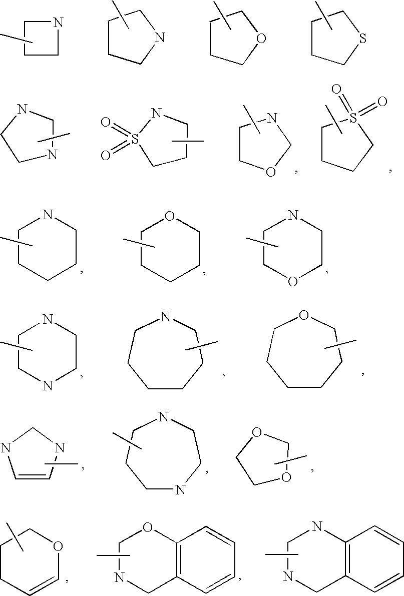

and the like, which optionally may be substituted at any available carbon or nitrogen atom.

The term “heteroaryl” refers to optionally substituted aromatic 5 to 7 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring.

Examples of heteroaryl rings include

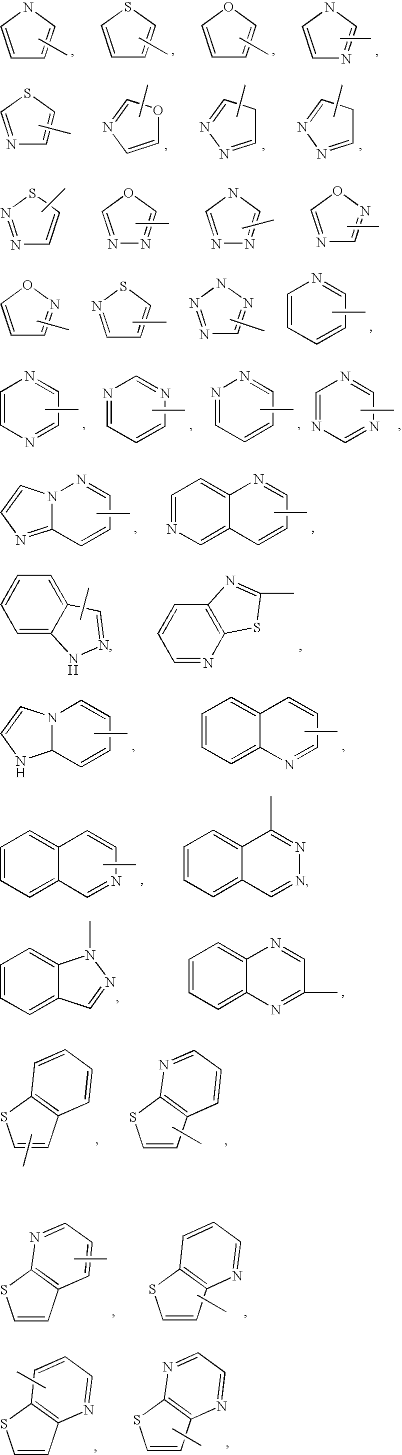

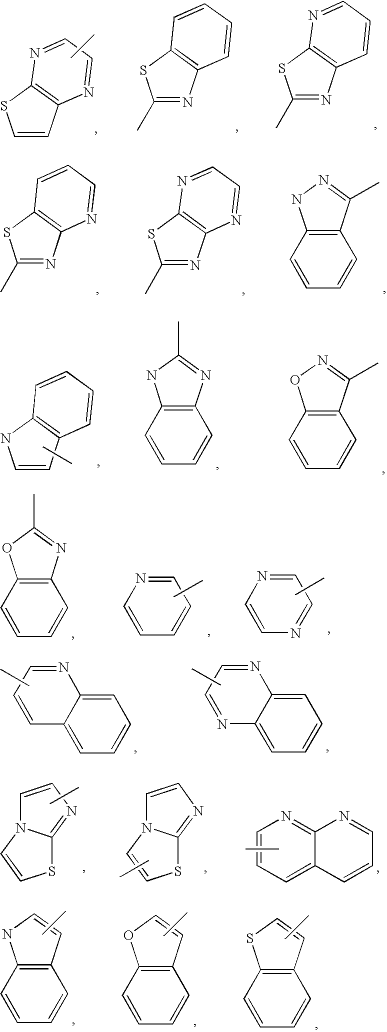

and the like, which optionally may be substituted at any available carbon or nitrogen atom. The term “pyridyl” also includes optionally substituted pyridyl.

The term “heterocyclic” or “heterocyclic ring” includes both heterocyclo and heteroaryl groups, as defined above.

When the term “unsaturated” is used herein to refer to a ring or group, the ring or group may be fully unsaturated or partially unsaturated.

It should be understood that one skilled in the art may make various substitutions for each of the groups recited in the claims herein, without departing from the spirit or scope of the invention.

Throughout the specification, groups and substituents thereof may be chosen by one skilled in the art to provide stable moieties and compounds.

The compounds of formula I through XV form salts which are also within the scope of this invention. Unless otherwise indicated, reference to an inventive compound is understood to include reference to salts thereof. The term “salt(s)” denotes acidic and/or basic salts formed with inorganic and/or organic acids and bases. In addition, the term “salt(s)” may include zwitterions (inner salts), e.g., when a compound of formula I through XII contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula I through XII may be formed, for example, by reacting the compound with an amount of acid or base, such as an equivalent amount, in a medium such as one in which the salt precipitates or in an aqueous medium followed by lyophilization.

Prodrugs and solvates of the inventive compounds are also contemplated. The term “prodrug” denotes a compound which, upon administration to a subject, undergoes chemical conversion by metabolic or chemical processes to yield a compound of the formula I, and/or a salt and/or solvate thereof. Various forms of prodrugs are well known in the art. For examples of such prodrug derivatives, see:

a) Design of Prodrugs, edited by H. Bundgaard, (Elsevier, 1985) and Methods in Enzymology, Vol.42, p. 309-396, edited by K. Widder, et al. (Acamedic Press, 1985);

b) A Textbook of Drug Design and Development, edited by Krosgaard-Larsen and H. Bundgaard, Chapter 5, “Design and Application of Prodrugs,” by H. Bundgaard, p. 113-191 (1991); and

c) H. Bundgaard, Advanced Drug Delivery Reviews, 8, 1-38 (1992), each of which is incorporated herein by reference.

Compounds of formula I through XV and salts thereof may exist in their tautomeric form, in which hydrogen atoms are transposed to other parts of the molecules and the chemical bonds between the atoms of the molecules are consequently rearranged. It should be understood that the all tautomeric forms, insofar as they may exist, are included within the invention. Additionally, inventive compounds may have trans and cis isomers and may contain one or more chiral centers, therefore existing in enantiomeric and diastereomeric forms.

This invention includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers or diastereomers as starting materials. When enantiomeric or diastereomeric products are prepared, they can be separated by conventional methods, for example chromatographic or fractional crystallization.

The compounds of the instant invention may, for example, be in the free or hydrate form.

The process of the instant invention is readily carried out as described below.

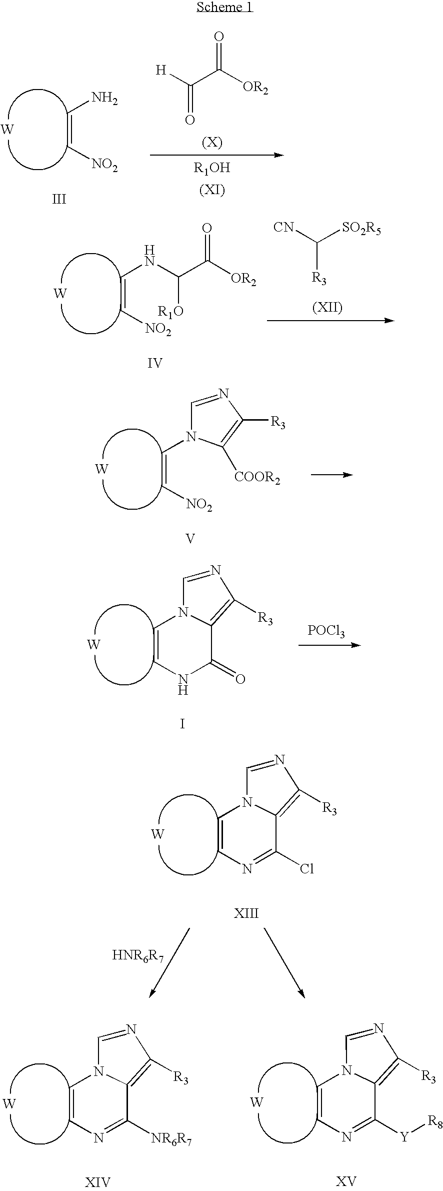

According to Scheme 1, a compound of formula III, wherein W is defined as above, reacts with a compound of formula X and a compound of formula XI, wherein R₂ and R₁ are independently C₁-C₄ alkyl, to produce a compound of formula IV. This reaction can be carried out at a temperature from about 0° C. to about 165° C. for from 15 minutes to about 48 hours. The preferred reaction temperature is from ambient temperature to about 150° C. The more preferred temperature is from 40° C. to about 120° C.

The compound of formula IV further reacts with a compound of formula XII, wherein R₃ is selected from the group consisting of H, C₁-C₄ alkyl, cycloalkyl, heterocyclo, aryl and heteroaryl, and R₅ is aryl, in the presence of a base to produce a compound of formula V.

A compound of formula I is obtained by reacting the compound of formula V with a reducing reagent selected from the group consisting of iron, zinc, sodium hydrosulfite and sodium hydrosulfite hydrate, in the presence of an acid with heating. This reaction has a distinct advantage of combining the reduction and cyclization in one step by using the selected reducing reagents. The reaction can be carried out at a temperature from about 40° C. to about 185° C. for from 5 minutes to about 48 hours. The preferred reaction temperature is from 60° C. to about 165° C. The more preferred temperature is from 80° C. to about 150° C.

A compound of formula XIV, wherein W and R₃ are defined as above; R₆ and R₇ are independently H, alkyl, cycloalkyl, heterocyclo, aryl, heteroaryl, or R₆ and R₇ together with the N to which they are bonded optionally form a heterocyclic ring, is prepared from the compound of formula I according to methods described U.S. Pat. No. 6,235,740.

A compound of formula XV, wherein W and R₃ are defined as above; Y is a bond, O, or S; R₈ is alkyl, alkenyl, alkynyl, cycloalkyl, heterocyclo, aryl, or heteroaryl, is prepared from the compound of formula I according to methods described in U.S. Pat. No. 6,239,133.

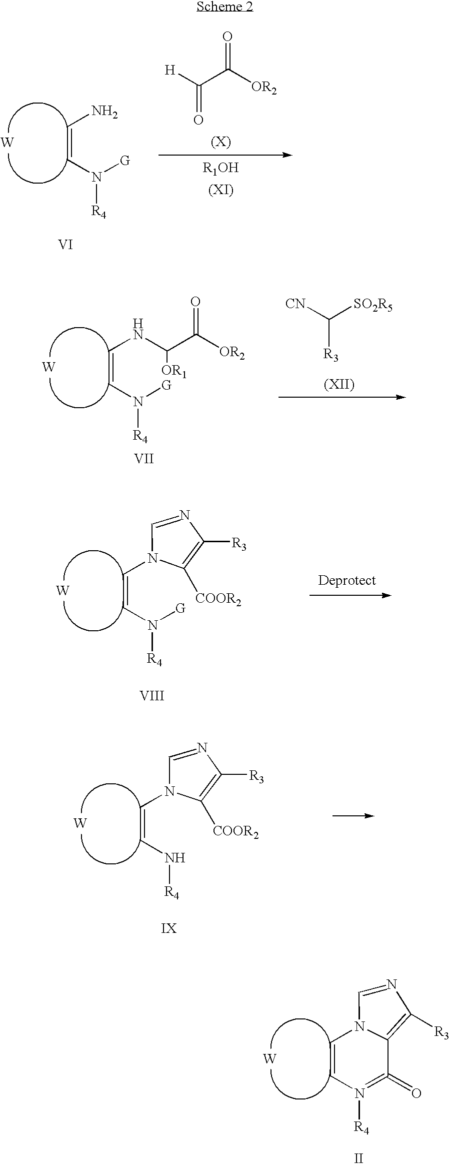

According to Scheme 2, a compound of formula VI, wherein W is as defined above, R₄ is H, alkyl, cycloalkyl, heterocyclo, aryl, or heteroaryl, G is an amine protecting group, reacts with a compound of formula X and a compound of formula XI, wherein R₂ and R₁ are independently C₁-C₄ alkyl, to produce a compound of formula VII. Any amine protecting group known to those skilled in the art can be used as G according to Scheme 2. The preferred protecting groups are Boc, CBZ, and benzyl. The more preferred protecting group is Boc. This reaction can be carried out at a temperature from about 0° C. to about 165° C. for from 15 minutes to about 48 hours. The preferred reaction temperature is from ambient temperature to about 150° C. The more preferred temperature is from 40° C. to about 120° C.

The compound of formula VII further reacts with a compound of formula XII, wherein R₃ and R₅ are as defined above, in the presence of a base to produce a compound of formula VIII.

A compound of formula IX is obtained from the compound of formula VIII by performing deprotection procedures well known to those skilled in the art. When G is CBZ, for example, the CBZ group is removed by hydrogenation over a catalyst. An acid such as HCl or trifluoroacetic acid may be added to the hydrogenation mixtrue to ensure complete reaction. It is preferred that a Pd catalyst is used in the removal of CBZ group.

Alternatively, when G is Boc, which is the more preferred protecting group, the compound of formula VIII may be conventionally treated with an acid to effect deprotection. Peferred acids for such deprotections include trifluroacetic acid, methanesulfonic acid and hydrochloric acid. A more preferred acid is trifluroacetic acid.

A compound of formula II is obtained by treating the compound of formula IX with a base while heating. This reaction can be carried out at a temperature from about 40° C. to about 185° C. for from 5 minutes to about 48 hours. The preferred reaction temperature is from 60° C. to about 165° C. The more preferred temperature is from 80° C. to about 150° C.

The compounds of formula XIV and formula XV shown in Scheme 1 can also be prepared from the compound of formula II, wherein R₄ is H, according to methods described U.S. Pat. Nos. 6,235,740 and 6,239,133.

The compounds of the formula I and formula II prepared by the processes of the present invention are themselves pharmacologically active, or are compounds which may be further converted to pharmacologically active products. Compounds of the formula I, wherein W, together with the atoms to which it is bonded, is aryl, may be converted into imidazoquinoxalines such as are described U.S. Pat. Nos. 6,235,740 and 6,239,133. Compounds of the formula I, wherein W together with the atoms to which it is bonded is heteroaryl, may be converted into heterocyclic-substituted imidazopyrazines such as are described in U.S. Pat. No. 5,990,109.

The present invention is further described by the following examples which are illustrative only, and are in no way intended to limit the scope of the instant claims. All references referred to in this specification are incorporated by reference in their entirety.

Abbreviations

AcOH Acetic acid

aq. Aqueous

Bn Benzyl

Boc tert-butoxycarbonyl

CBZ benzoyloxycarbonyl

DCM dichloromethane

DI water de-ionized water

DMF dimethylformamide

DMSO Dimethylsulfoxide

EtOAc Ethyl acetate

Et Ethyl

EtOH Ethanol

HPLC High pressure liquid chromatography

LC liquid chromatography

Me Methyl

MeOH Methanol

min. Minutes

M⁺ (M+H)⁺

M⁺¹ (M+H)⁺

MS Mass spectrometry

n normal

Pd/C Palladium on carbon

Ph Phenyl

Ret Time Retention time

rt or RT Room temperature

sat'd Saturated

TFA Trifluoroacetic acid

THF Tetrahydrofuran

TOSMIC Tosylmethyl isocyanide

### EXAMPLE 1

### Preparation of Preparation of 7,8-Dimethoxy-imidazoquinoxalin-4-one from 2-nitro-4,5-dimethoxyaniline

1A. Preparation of Ethyl α-Methoxy-α-(2-Nitro-4,5-dimethoxyphenylamino)acetate

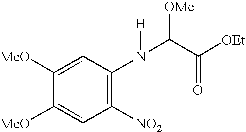

To a 500 mL round-bottom flask was added 10.00 g (50.46 mmol) of 2-nitro-4,5-dimethoxyaniline, 200 mL of anhydrous methanol and 50 mL (252.2 mmol) of ethyl glyoxylate solution (50 wt % in toluene). The suspension was heated to reflux and stirred under argon for 16 hrs. A Dean-Stark trap was added to the apparatus and about 100 mL of distillate were removed. An additional 100 mL of anhydrous methanol were added and the reaction mixture was refluxed for 30 minutes while an additional 105 mL of distillate were removed using the Dean Stark trap. An additional 10 mL ethyl glyoxylate solution and 100 mL of anhydrous methanol were added and the reaction was refluxed for another 1.3 hrs while removing another 15 mL of distillate. The reaction mixture was allowed to cool to room temperature and stirred overnight under argon. The suspended crystals were isolated by filtration and were washed with ˜20 mL of methanol and with ˜20 mL of heptane. The wet cake was dried in vacuo at ˜40-45° C. to give 13.97 g (88.1%) of ethyl α-methoxy-α-(2-nitro-4,5-dimethoxyphenylamino)acetate. ¹H NMR: (CDCl₃) δ 1.36 (t, J=7.2 Hz, 3H), 3.34 (s, 3H), 3.88 (s, 3H), 3.95 (s, 3H), 4.35 (q, J=7.2 Hz, 2H), 5.35 (d, J=5.8 Hz, (1H), 6.59 (s, 1H), 7.67 (s, 1H), 9.13 (d, J=5.8 Hz, 1H).

1B. Preparation of Ethyl 1-(2-Nitro-4,5-dimethoxyphenyl)-imidazole-5-carboxylate

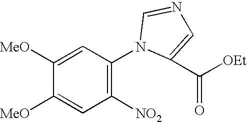

A To a 2L round-bottom flask was added 32.95 g (104.84 mmol) of ethyl α-methoxy-α-(2-nitro-4,5-dimethoxyphenylamino)acetate, 27.04 g (138.50 mmol) of tosylmethyl isocyanide, 1.44L of absolute ethanol. The reaction mixture was stirred under argon and 36.15 g (261.56 mmol) of potassium carbonate were added. The suspension was heated to ˜50° C. and stirred for ˜3.75 hrs. The reaction mixture was then concentrated in vacuo at ˜50° C. The resulting residue was slurried in water. The suspended solids were isolated by filtration and were washed with water. The wet cake was slurried in 50 mL of 2-propanol and then 100 mL heptane. The wet cake was dried at 45° C. to give 23.00g of ethyl 1-(2-nitro-4,5-dimethoxyphenyl)-imidazole-5-carboxylate. (CDCl₃) δ 7.87 (s, 1H), 7.78 (s, 1H), 7.67 (s, 1H), 6.80 (s, 1H), 4.18 (q, J=7.1 Hz, 2H), 4.03 (s, 3H), 3.97 (s, 3H), 1.25 (t, J=7.1 Hz, 3H).

1C(a). Preparation of 7,8-Dimethoxy-imidazoquinoxalin-4-one

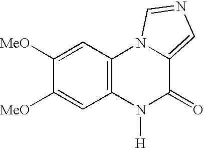

To a 250 mL round-bottom flask was added 5.0g (15.56 mmol) of ethyl 1-(2-nitro-4,5-dimethoxyphenyl)-imidazole-5-carboxylate, 50 mL of glacial acetic acid and 4.4 g (78.78 mmol) iron powder. The mixture was heated to 105° C. and stirred for 30 minutes under nitrogen. 150 mL of DI water was added and the slurry was cooled to room temperature. After stirring for 3 hours, the slurry was filtered. The cake was washed with 6×50 mL DI water and dried in a vacuum oven at 45° C. for 18 hours. The solid was dissolved with 40 mL DMF at 105° C. and filtered to remove residual iron metal. The filtrate was cooled to 50° C. and 150 mL of DI water was added to the resulted slurry. After cooling to room temperature and stirring for 3 hours, the slurry was filtered, washed with 3×20 mL DI water and dried in a vacuum oven at 45° C. for 19 hours to give 3.59 g of 7,8-dimethoxy-imidazoquinoxalin-4-one. ¹H NMR: (DMSO-d₆) δ 3.79 (s, 3H), 3.87 (s, 3H), 6.90 (s, 1H), 7.79 (s, 1H), 7.81 (s, 1H), 9,02 (s, 1H).

1C(b) Preparation of 7,8-Dimethoxy-imidazoquinoxalin-4-one

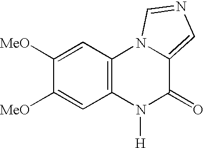

This is an alternate method to the method described in 1C(a) above. To a 1L round-bottom flask was added 19.36 g (60.26 mmol) of ethyl 1-(2-nitro-4,5-dimethoxyphenyl)-imidazole-5-carboxylate, 100 mL of glacial acetic acid and 100 mL of DI water. The mixture was stirred to give a slurry and 41.97 g (241.03 mmol) of sodium dithionite was added. The reaction mixture was heated to 105° C. and stirred for 6 hours under nitrogen. 500 mL of DI water was added and the slurry was cooled to room temperature. After stirring for 2 hours, the slurry was filtered. The cake was washed with 3×50 mL DI water and dried in a vacuum oven at 45° C. for 42 hours to give 16.43 g of 7,8-dimethoxy-imidazoquinoxalin-4-one. ¹H NMR: (DMSO-d₆) δ 3.79 (s, 3H), 3.87 (s, 3H), 6.90 (s, 1H), 7.79 (s, 1H), 7.81 (s, 1H), 9,02 (s, 1H)

### EXAMPLE 2

### Preparation of 7-Chloroimidazo[1,5-a]pyrido[4,3-e]pyrazine-4(5H)-one from (5-Amino-2-chloro-4-pyridinyl)carbamic acid 1,1-dimethylethyl ester

2A. Preparation of 2-[[6-Chloro-4-[[(1,1-dimethylethoxy)carbonyl]amino]-2-pyridinyl]amino]-2-methoxyacetic acid ethyl ester

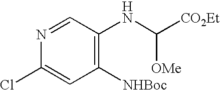

A mixture of (5-Amino-2-chloro-4-pyridinyl)carbamic acid 1,1-dimethylethyl ester (300 mg, 1.23 mmol) and ethyl glyoxylate (50% solution in toluene, 0.32 mL, 1.60 mmol) in MeOH was heated at 65° C. for 5 hrs. The reaction mixture was diluted with MeOH and the resulting precipitate was removed by filtration. The filtrate was concentrated in vacuo to give 435 mg of 2-[[6-Chloro-4-[[(1,1-dimethylethoxy)carbonyl]amino]-2-pyridinyl]amino]-2-methoxyacetic acid ethyl ester as a yellow oil. ¹H NMR (CDCl₃) δ 1.35 (t, J=7.1 Hz, 3H), 3.50 (s, 3H), 4.30 (q, J=7.1 Hz, 2H), 5.28 (s, 1H), 7.86 (s, 1H), 8.03 (s, 1H), 8.08 (s, 1H), 8.28 (s, 1H).

2B. Preparation of 3-[6-Chloro-4-[[(1,1-dimethylethoxy)carbonyl]amino]-2-pyridinyl]-3H-imidazole-4-carboxylic acid ethyl ester

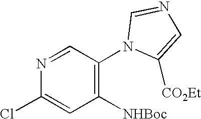

A mixture of 2-[[6-Chloro-4-[[(1,1-dimethylethoxy)carbonyl]amino]-2-pyridinyl]amino]-2-methoxyacetic acid ethyl ester (435 mg, 1.22 mmol), tosylmethyl isocyanide (592 mg, 3.04 mmol) and solid K₂CO₃ (670 mg, 4.86 mmol) in 11 mL of EtOH was heated at 60° C. for 3 hrs. Water was added and the beige precipitate was collected by filtration, rinsed with more water. Drying under high vacuum gave 430 mg of 3-[6-Chloro-4-[[(1,1-dimethylethoxy)carbonyl]amino]-2-pyridinyl]-3H-imidazole-4-carboxylic acid ethyl ester. ¹H NMR (CDCl₃) δ 1.25 (t, J=7.1 Hz, 3H), 1.49 (s, 9H), 4.23 (q, J=7.1 Hz, 2H), 6.22 (s, 1H), 7.65 (s, 1H), 7.98 (s, 1H), 8.07 (s, 1H), 8.35 (s, 1H).

2C. Preaparation of 7-Chloroimidazo[1,5-a]pyrido[4,3-e]pyrazine-4(5H)-one

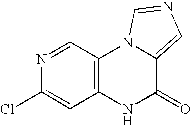

A mixture of 3-[6-Chloro-4-[[(1,1-dimethylethoxy)carbonyl]amino]-2-pyridinyl]-3H-imidazole-4-carboxylic acid ethyl ester (430 mg) and trifluoroacetic acid (3 mL) was stirred for 15 min. Concentration in vacuo and the residue was taken in CH₂Cl₂, washed with Sat'd NaHCO₃, water, sat'd NaCl and dried over anhydrous Na₂SO₄. Flash chromatography (Hexane/EtOAc: 3:1) on silica gel gave 223 mg of intermediate as a yellow solid.

A mixture of above intermediate (830 mg, 3.12 mmol) and solid K₂CO₃ (760 mg, 5.5 mmol) in 35 mL of dry DMF was heated to reflux for 1.0 hr. Concentration in vacuo and followed by addition of AcOH to the residue (pH 7.0). The precipitate was collected by filtration, rinsed with water and dried under high vacuum to give 568 mg of 7-Chloroimidazo[1,5-a]pyrido[4,3-e]pyrazine-4(5H)-one as a beige solid. ¹H NMR (CD₃OD) δ 5.48 (s, 1H), 7.26 (s, 1H), 7.97 (s, 1H), 9.01 (s, 1H), 9.10 (s, 1H).

What is claimed is:

1. A process for preparing a compound of formula I, 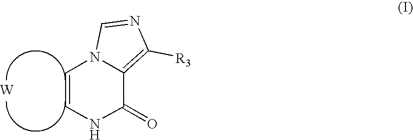
wherein
W, together with the atoms to which it is bonded, is a 4-15 membered monocyclic or bicyclic ring system optionally including up to 4 heteroatoms selected from N, O or S, and wherein a carbon atom in the said ring system is optionally substituted with oxo, and wherein W is optionally substituted with 1-3 substituents selected from the group consisting of halogen, trifluoromethyl, trifluoromethoxy, alkyl, alkenyl, alkynyl, cyano, ORₐ, SRₐ, NR_{b}R_{c}, NR_{b}SO₂Rₐ, SO₂Rₐ, SO₂NR_{b}R_{c}, CO₂Rₐ, C(═O)Rₐ, C(═O)NR_{b}R_{c}, OC(═O)Rₐ, OC(═O)NR_{b}R_{c}, NR_{b}C(═O)ORₐ, NR_{d}C(═O)NR_{b}R_{c}, NR_{b}C(═O)Rₐ, cycloalkyl, heterocyclo, aryl, and heteroaryl, wherein Rₐ is alkyl, alkenyl, alkynyl, cycloalkyl, heterocyclo, aryl or heteroaryl, R_{b}, R_{c}, and R_{d} are independently H, alkyl, cycloalkyl, heterocyclo, aryl, or heteroaryl, said R_{b} and R_{c} together with the N to which they are bonded optionally form a heterocyclic ring;
R₃ is selected from the group consisting of H, C₁-C₄ alkyl, cycloalkyl, heterocyclo, aryl and heteroaryl;
comprising reacting a compound of Formula V, 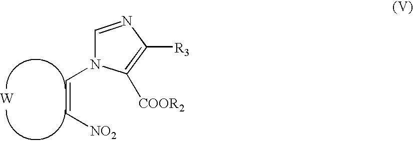
wherein W and R₃ are as defined hereinabove, and R₂ is C₁-C₄ alkyl,
with a reducing reagent selected from the group consisting of iron, zinc, sodium hydrosulfite and sodium hydrosulfite hydrate, in the presence of an acid, with heating.

2. The process of claim 1, wherein W is aryl or heteroaryl.

3. The process of claim 1, wherein W is phenyl.

4. The process of claim 1, wherein W is pyridyl.

5. The process of claim 1, further comprising converting the compound of formula I to a compound of formula XIV, 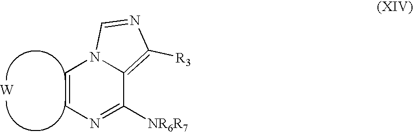
wherein W and R₃ are defined as in claim 1; R₆ and R₇ are independently H, alkyl, cycloalkyl, heterocyclo, aryl, heteroaryl, or R₆ and R₇ together with the N to which they are bonded optionally form a heterocyclic ring.

6. The process of claim 1, further comprising converting the compound of formula I to a compound of formula XV, 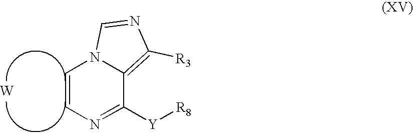
wherein W and R₃ are defined as in claim 1; Y is a bond, O, or S; R₈ is alkyl, alkenyl, alkynyl, cycloalkyl, heterocyclo, aryl, or heteroaryl.

7. A process for preparing a compound of formula I, 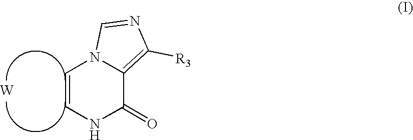
wherein
W, together with the atoms to which it is bonded, is a 4-15 membered monocyclic or bicyclic ring system optionally including up to 4 heteroatoms selected from N, O or S, and wherein a carbon atom in the said ring system is optionally substituted with oxo, and wherein W is optionally substituted with 1-3 substituents selected from the group consisting of halogen, trifluoromethyl, trifluoromethoxy, alkyl, alkenyl, alkynyl, cyano, ORₐ, SRₐ, NR_{b}R_{c}, NR_{b}SO₂Rₐ, SO₂Rₐ, SO₂NR_{b}R_{c}, CO₂Rₐ, C(═O)Rₐ, C(═O)NR_{b}R_{c}, OC(═O)Rₐ, OC(═O)NR_{b}R_{c}, NR_{b}C(═O)ORₐ, NR_{d}C(═O)NR_{b}R_{c}, NR_{b}C(═O)Rₐ, cycloalkyl, heterocyclo, aryl, and heteroaryl, wherein Rₐ is alkyl, alkenyl, alkynyl, cycloalkyl, heterocyclo, aryl or heteroaryl, R_{b}, R_{c} and R_{d} are independently H, alkyl, cycloalkyl, heterocyclo, aryl, or heteroaryl, said R_{b} and R_{c} together with the N to which they are bonded optionally form a heterocyclic ring;
R₃ is selected from the group consisting of H, C₁-C₄ alkyl, cycloalkyl, heterocyclo, aryl and heteroaryl;
comprising:
(a) reacting a compound of formula III, 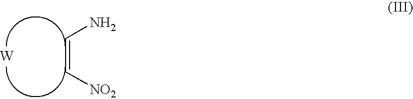
wherein W is as defined hereinabove,
with a compound of formula X, 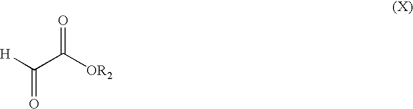
wherein R₂ is C₁-C₄ alkyl,
and a compound of formula XI,
R₁OH (XI)
wherein R₁ is C₁-C₄ alkyl.
to produce a compound of formula IV; 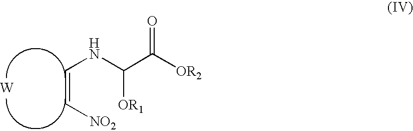
(b) reacting the compound of formula IV with a compound of formula XII, 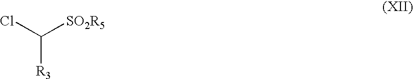
wherein R₃ is as defined hereinabove, and R₅ is aryl, in the presence of a base to produce a compound of formula V; and 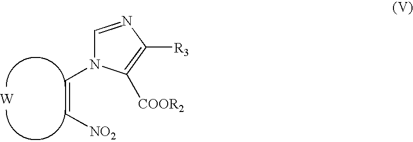
(c) reacting the compound of formula V with a reducing reagent selected from the group consisting of iron, zinc, sodium hydrosulfite and sodium hydrosulfite hydrate, in the presence of an acid, with heating.

8. The process of claim 7, wherein W is aryl or heteroaryl.

9. The process of claim 7, wherein W is phenyl.

10. The process of claim 7, wherein W is pyridyl.

11. The process of claim 7, further comprising converting the compound of formula I to a compound of formula XIV, 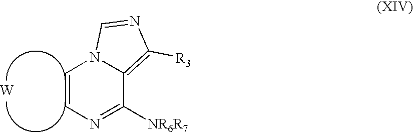
wherein W and R₃ are defined as in claim 7; R₆ and R₇ are independently H, alkyl, cycloalkyl, heterocyclo, aryl, heteroaryl, or R₆ and R₇ together with the N to which they are bonded optionally form a heterocyclic ring.

12. The process of claim 7, further comprising converting the compound of formula I to a compound of formula XV, 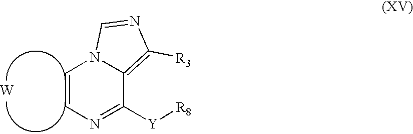
wherein W and R₃ are defined as in claim 7; Y is a bond, O, or S; R₈ is alkyl, alkenyl, alkynyl, cycloalkyl, heterocyclo, aryl, or heteroaryl.

13. A process for preparing a compound of formula II, 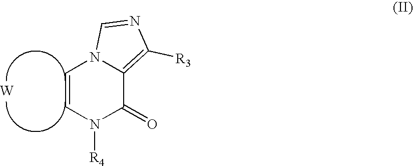
wherein
W, together with the atoms to which it is bonded, is a 4-15 membered monocyclic or bicyclic ring system optionally including up to 4 heteroatoms selected from N, O or S, and wherein a carbon atom in the said ring system is optionally substituted with oxo, and wherein W is optionally substituted with 1-3 substituents selected from the group consisting of halogen, trifluoromethyl, trifluoromethoxy, alkyl, alkenyl, alkynyl, cyano, ORₐ, SRₐ, NR_{b}R_{c}, NR_{b}SO₂Rₐ, SO₂Rₐ, SO₂NR_{b}R_{c}, CO₂Rₐ, C(═O)Rₐ, C(═O)NR_{b}R_{c}, OC(═O)Rₐ, OC(═O)NR_{b}R_{c}, NR_{b}C(═O)ORₐ, NR_{d}C(═O)NR_{b}R_{c}, NR_{b}C(═O)Rₐ, cycloalkyl, heterocyclo, aryl, and heteroaryl, wherein Rₐ is alkyl, alkenyl, alkynyl, cycloalkyl, heterocyclo, aryl or heteroaryl, R_{b}, R_{c} and R_{d} are independently H, alkyl, cycloalkyl, heterocyclo, aryl, or heteroaryl, said R_{b} and R_{c} together with the N to which they are bonded optionally form a heterocyclic ring;
R₃ and R₄ are, independently, H, alkyl, cycloalkyl, heterocyclo, aryl, or heteroaryl;
comprising:
(a) reacting a compound of formula VI, 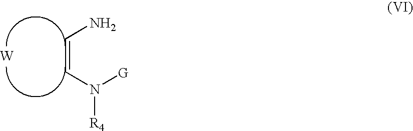
wherein W and R₄ are as defined hereinabove, G is an amine protecting group,
with a compound of formula X, 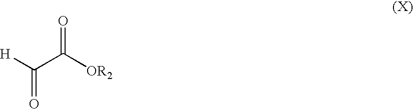
wherein R₂ is C₁-C₄ alkyl,
and a compound of formula XI,
R₁OH (XI)
wherein R₁ is C₁-C₄ alkyl,
to produce a compound of formula VII; 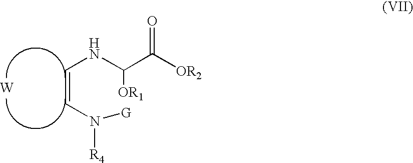
(b) reacting the compound of formula VII with a compound of formula XII, 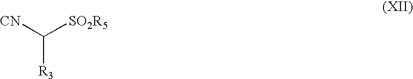
wherein R₃ is as defined hereinabove, and R₅ is aryl, in the presence of a base to produce a compound of formula VIII; and 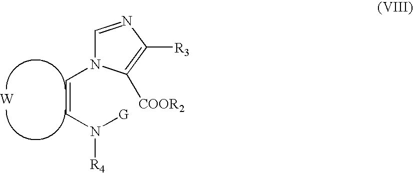
(c) deprotecting the compound of formula VIII to give a compound of formula of IX; and 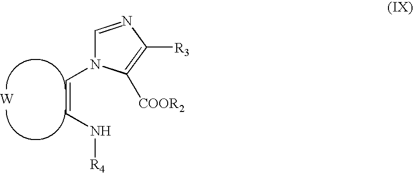
(d) reacting the compound of formula IX with a base while heating.

14. The process of claim 13, wherein W is aryl or heteroaryl.

15. The process of claim 13, wherein W is phenyl.

16. The process of claim 13, wherein W is pyridyl.

17. The process of claim 13, further comprising converting the compound of formula II wherein R₄ is H, to a compound of formula XIV, 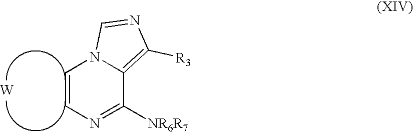
wherein W and R₃ are defined as in claim 13; R₆ and R₇ are independently H, alkyl, cycloalkyl, heterocyclo, aryl, heteroaryl, or R₆ and R₇ together with the N to which they are bonded optionally form a heterocyclic ring.

18. The process of claim 13, further comprising converting the compound of formula II wherein R₄ is H, to a compound of formula XV, 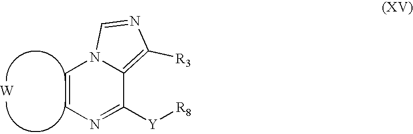
wherein W and R₃ are defined as in claim 13; Y is a bond, O, or S; R₈ is alkyl, alkenyl, alkynyl, cycloalkyl, heterocyclo, aryl, or heteroaryl.
